(19) United States
(12) Patent Application Publication
(10) Pub. No.: US 2015/0105806 A1
(43) Pub. Date: Apr. 16, 2015

(54) Universal Cranioplasty Mesh

(71) Applicants: Amir Hossein Dorafshr, Baltimore, MD (US); Axel Waizenegger, Muhlheim (DE); Siegbert Schilling, Kolbingen (DE); Patrick Lemoine, Jacksonville Beach, FL (US)

(72) Inventors: Amir Hossein Dorafshr, Baltimore, MD (US); Axel Waizenegger, Muhlheim (DE); Siegbert Schilling, Kolbingen (DE); Patrick Lemoine, Jacksonville Beach, FL (US)

(21) Appl. No.: 14/391,488

(22) PCT Filed: Apr. 9, 2013
(86) PCT No.: PCT/US13/35727

§ 371 Date: Oct. 9, 2014

Related U.S. Application Data
(60) Provisional application No. 61/621,609, filed on Apr. 9, 2012.

(51) Int. Cl. A61F 2/00

(52) U.S. Cl. CPC A61F 2/0063; A61F 2002/0068
USPC 606/151

(57) ABSTRACT

An embodiment in accordance with the present invention provides a device for use in a cranioplasty including a first mesh, wherein a structure of the first mesh has a generally rigid, contoured configuration. The device also includes a second mesh, wherein a structure of the second mesh has a generally malleable, contoured configuration. The first mesh and the second mesh are also seamlessly integrated into a hybrid mesh. This design allows for rigid structure in certain regions of the implant and customization and malleability in other regions.

### CROSS REFERENCE TO RELATED APPLICATIONS

This application claims the benefit of U.S. Provisional Patent Application No. 61/621,609 filed on Apr. 9, 2012, which is incorporated by reference, herein, in its entirety.

### FIELD OF THE INVENTION

The present invention relates generally to surgery. More particularly, the present invention relates to a device for performing a cranioplasty.

### BACKGROUND OF THE INVENTION

Cranioplasty is a surgical procedure to repair a defect or deformity of a skull. Different methods of performing cranioplasty can be used, such as bone graft, plastic implants, and metal implants.

One type of metal implant used is a flat titanium mesh. This mesh can be bent somewhat to follow the shape of the skull. However, in some cases, these flat meshes can be hard to bend and do not necessarily remain in the bent position. Additionally, it can be time consuming and technically demanding to bend the mesh to the desired configuration.

In order to provide a better fit to the patient's skull, a pre-formed mesh can be used. Often these pre-formed mesh pieces are designed for particular regions of the skull. Unfortunately, that means that some areas of the skull are not represented in these pre-formed pieces, especially with respect to pieces that can be used to reconstruct the orbital roof, the nose, the occipital portion of the skull, and the temporal region. These pre-formed pieces also include countersunk fittings for screws around an outside edge of the piece. However, if these pre-formed pieces are then cut down to cover a smaller region of the skull, these countersunk fittings around the edges are lost.

To provide a customized fit for the injury or deformity, a custom mesh can be made to fit the exact shape, size and curvature of the patient's skull. A three-dimensional model of the patient's skull is taken and the implant piece or pieces are custom fit to the shape and size of the model. This solution can, however, be time consuming to produce and quite costly given the custom nature of the implant. Additionally, this is not necessarily a viable solution for a trauma situation, in which time is of the essence.

It would therefore be advantageous to provide a mesh that is generally pre-formed from a hybrid mesh with regions that can be easily and quickly customized to a patient's unique cranial anatomy.

### SUMMARY OF THE INVENTION

The foregoing needs are met, to a great extent, by the present invention, wherein in one aspect a device for use in cranioplasty includes a first mesh. A structure of the first mesh has a generally rigid configuration. The device also includes a second mesh, and a structure of the second mesh has a generally malleable configuration. The first mesh and the second mesh are seamlessly integrated into a hybrid mesh.

In accordance with an aspect of the present invention, the first mesh and the second mesh are formed from titanium. The first mesh is pre-formed to have a fixed shape conforming to predetermined regions of a skull. The second mesh is malleable, such that it conformable to predetermined regions of a skull. The first mesh is configured to couple with a screw for securing the first mesh to a skull, and the first mesh is configured such that the screw is countersunk within the first mesh. In addition, the device is configured such that the second mesh is moldable to cover the orbital rim and roof, nose, temporal, and occiput of a skull. The device can have four component pieces, and can also include bridge regions to connect the four component pieces to one another. The second mesh is configured to couple with a screw for securing the second mesh to a skull, and the second mesh can be configured such that the screw is countersunk within the second mesh.

In accordance with another aspect of the present invention, a system for cranioplasty of a skull includes a cranioplasty component configured for covering at least a portion of a skull of a patient. The cranioplasty component is formed from a first mesh wherein a structure of the first mesh has a generally rigid configuration and a second mesh, wherein a structure of the second mesh has a generally malleable configuration. The first mesh and the second mesh are seamlessly integrated into a hybrid mesh. The system also includes a fastener configured to secure the cranioplasty component to the skull and a fastener receiving portion configured such that the fastener is disposable within to secure the cranioplasty component to the skull.

In accordance with yet another aspect of the present invention, the cranioplasty component includes multiple cranioplasty components. Each of these cranioplasty components are configured to cover a region of the skull. The multiple cranioplasty components are pre-connected to one another and can be coupled using a fastener. The fasteners are countersunk within the fastener receiving portions. Additionally, the multiple cranioplasty components comprise bridge regions for coupling the multiple cranioplasty components. The first mesh and the second mesh are formed from titanium. The first mesh can also be pre-formed to have a fixed shape conforming to predetermined regions of a skull.

### BRIEF DESCRIPTION OF THE DRAWINGS

The accompanying drawings provide visual representations which will be used to more fully describe the representative embodiments disclosed herein and can be used by those skilled in the art to better understand them and their inherent advantages. In these drawings, like reference numerals identify corresponding elements and:

FIG. 1 illustrates a frontal view of a hybrid mesh according to an embodiment of the invention.

FIG. 2 illustrates a top-down view of the hybrid mesh illustrated in FIG. 1 according to an embodiment of the invention.

FIG. 3 illustrates a left-side view of the hybrid mesh illustrated in FIGS. 1 and 2 according to an embodiment of the invention.

FIG. 4 illustrates a right-side view of the hybrid mesh illustrated in FIGS. 1-3 according to an embodiment of the invention.

FIG. 5 illustrates a partial view of a rigid contoured mesh according to an embodiment of the invention.

FIG. 6 illustrates a partial view of a malleable, contoured configuration for areas where molding is necessary according to an embodiment of the invention.

FIG. 7 illustrates an exploded view of four component pieces according to an embodiment of the invention.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

An embodiment in accordance with the present invention provides a device for use in a cranioplasty including a first mesh, wherein a structure of the first mesh has a generally rigid, contoured configuration. The device also includes a second mesh, wherein a structure of the second mesh has a generally malleable, contourable configuration. The first mesh and the second mesh are also seamlessly integrated. This design allows for rigid structure in certain regions of the implant and customization and malleability in other regions.

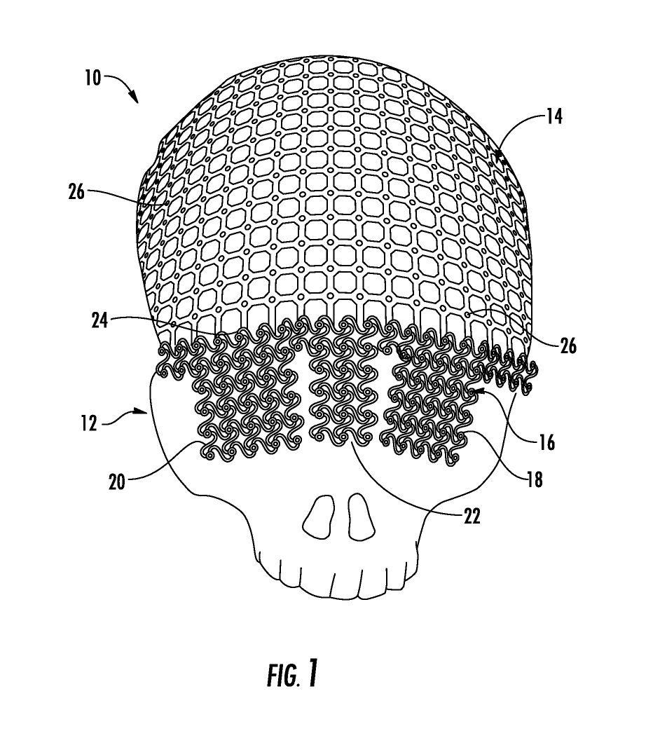

FIG. 1 illustrates a frontal view of a hybrid mesh according to an embodiment of the invention. As illustrated in FIG. 1, a mesh device 10 for use in a cranioplasty surgery is configured to fit operatively on a skull 12. The mesh device can include a rigid, contoured mesh 14 and a malleable, contoured mesh 16. Both the rigid, contoured mesh 14 and the malleable, contoured mesh 16 are formed from a metal or metal alloy, and preferably are formed from titanium. The rigid, contoured mesh 14 can be configured such that it is more rigid and less malleable than the malleable, contoured mesh 16. The rigid, contoured mesh 14 is pre-formed to fit the shape of an average skull. It is also possible that the device 10 can be manufactured in different sizes to further customize the fit to patients with different sized skulls. In some instances, therefore, the shape of the rigid, contoured mesh can be fixed, such that it is non-mutable, and can be utilized without first altering the shape. However, this configuration should not be considered limiting and any configuration of the rigid, contoured mesh known to or conceivable by one of skill in the art could also be used. The malleable, contoured mesh 16 should further be configured such that it can be bent and formed around more delicate and detailed areas of the skull 12, such as the orbital rim and roof, the nasal bridge, the temporal region, and the occipital portion of the skull.

Additionally, the rigid, contoured mesh 14 and the malleable, contoured mesh 16 are seamlessly integrated, in order to provide a smooth transition between the two types of mesh. For instance, the rigid, contoured mesh 14 and the malleable, contourable mesh can be manufactured at the same time, to ensure that the rigid, contoured mesh 14 transitions into the malleable, contoured mesh 16. The transition between the rigid contoured mesh 14 and the malleable, contourable mesh is illustrated in FIG. 1.

As shown in FIG. 1, there is a first portion 18 of the malleable, contoured mesh 16 for contouring into the orbital rim and roof of the left orbital region of the skull 12, a second portion 20 of the malleable, contoured mesh 16 for contouring into the orbital rim and roof of the right orbital region of the skull 12, and a third portion 22 of the malleable, contoured mesh 16 for contouring around the nasal bridge. A fourth portion 24 can also be used to more precisely form the mesh over the superior orbital rim. The device 10 also includes fittings 26 for screws (not shown) to secure the device 10 to the skull 12. Preferably, these fittings 26 are countersunk, such that the screws do not protrude significantly from the surface of the skull 12. The fittings 26 can be dispersed over the entire area of the device 10, throughout both the rigid, contoured mesh 14 and malleable, contoured mesh 16 regions, such that, if the device 10 is cut down to cover a smaller area of the skull 12 than the original device 10, there are still fittings 26, and more particularly, countersunk fittings for accepting screws to secure that smaller piece to the skull 12.

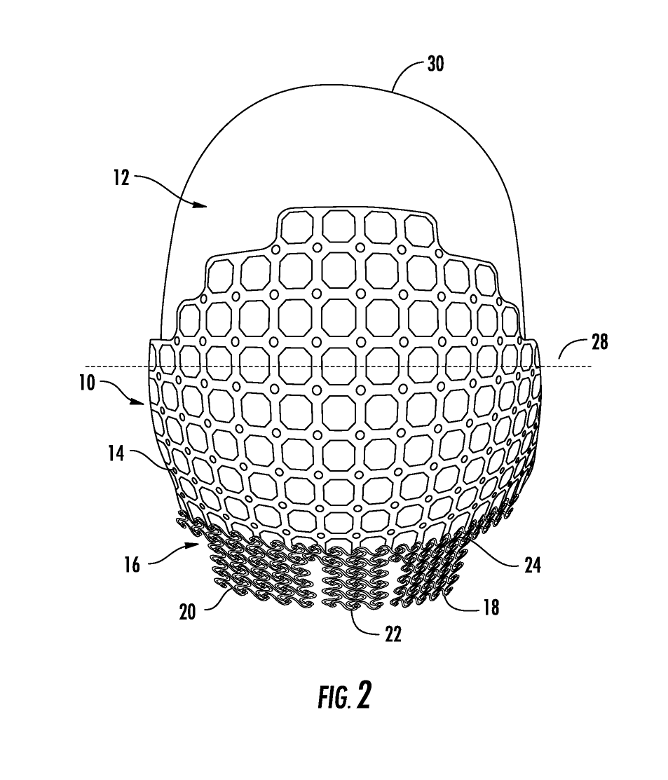

FIG. 2 illustrates a top-down view of the hybrid mesh illustrated in FIG. 1 according to an embodiment of the invention. As illustrated in FIG. 1, FIG. 2 shows the rigid, contoured mesh 14 and the malleable, contoured mesh 16. The first portion 18 of the malleable, contoured mesh 16 for contouring into the orbital rim and roof of the left orbital region of the skull 12, the second portion 20 of the malleable, contoured mesh 16 for contouring into the orbital rim roof of the right orbital region of the skull 12, and the third portion 22 of the malleable, contoured mesh 16 for contouring around the nasal bridge, can be seen in FIG. 2 from another angle. The fourth portion 24 can also be seen from this angle. While the rigid, contoured mesh 14 is illustrated here, as terminating just past a transverse midline 28 of the skull 12, the rigid, contoured mesh 14 could extend all the way to the occiput 30 of the skull 12.

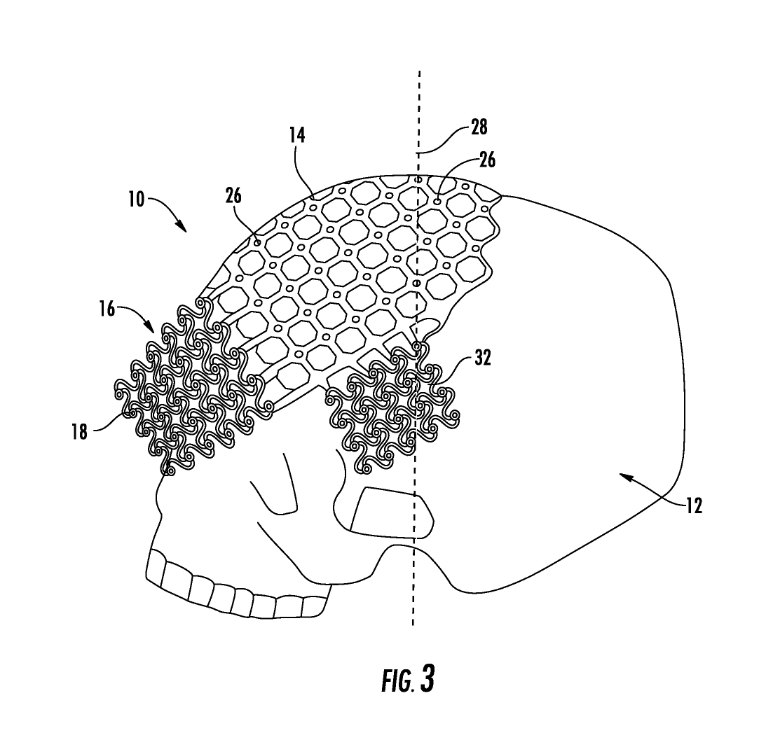

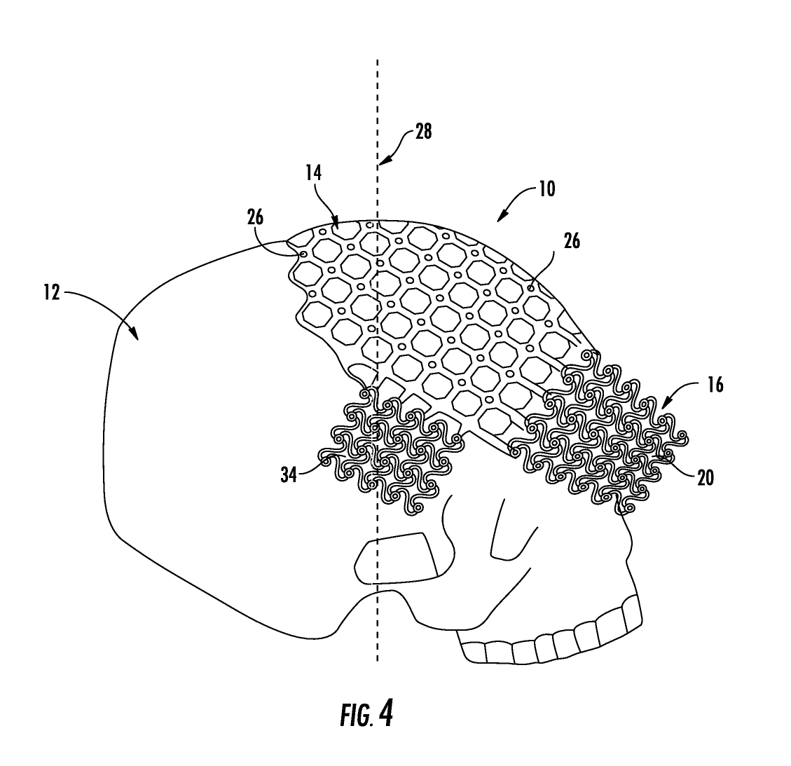

FIG. 3 illustrates a left-side view of the hybrid mesh illustrated in FIGS. 1 and 2 according to an embodiment of the invention and FIG. 4 illustrates a right-side view of the hybrid mesh illustrated in FIGS. 1-3 according to an embodiment of the invention. Both FIG. 3 and FIG. 4 illustrate the rigid, contoured mesh 14. FIG. 3 illustrates the first portion 18 of the malleable, contoured mesh 16 and a fifth portion 32 for contouring around the left temporal region of the skull 12. FIG. 4 illustrates the second portion 20 of the malleable, contoured mesh 16 and a sixth portion 34 for contouring around the right temporal region of the skull 12. Similarly to FIG. 2, while the device is shown terminating just past the transverse midline of the skull 12, the device 10 and the rigid, contoured mesh 14 and the malleable, contoured mesh 16 can be extended all the way around the skull 12, or in any other manner known to one of ordinary skill in the art.

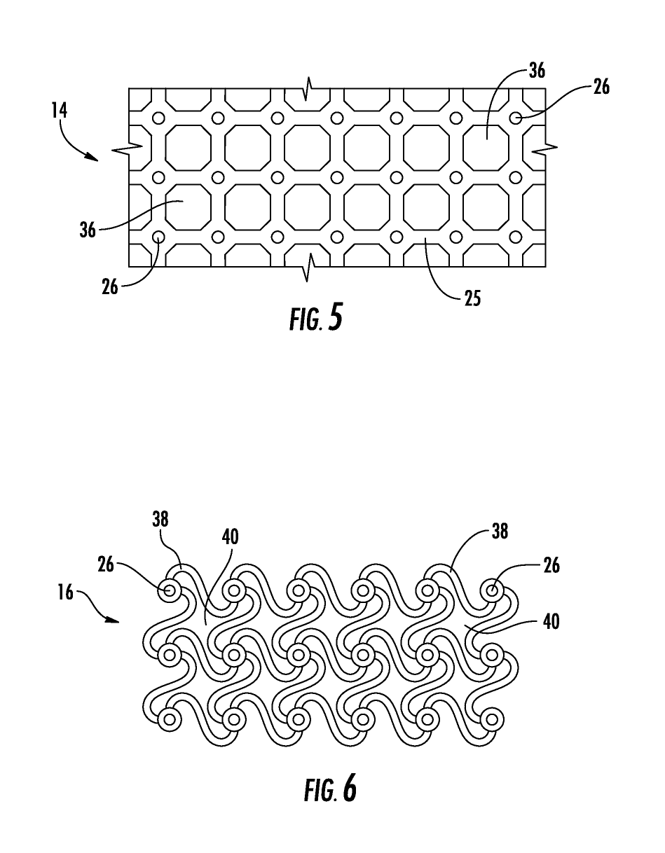

FIG. 5 illustrates a partial view of a rigid, contoured configuration rigid, contoured mesh according to an embodiment of the invention. As illustrated in FIG. 5, the rigid, contoured mesh 14 can be formed from a geometric pattern of areas 36 removed from the material used to form the mesh 14. While there is an exemplary pattern illustrated in FIG. 5, this pattern is not meant to be limiting. Any pattern known to one of skill in the art or suitable for covering the skull can be used. The rigid, contoured mesh 14 also includes the fittings 26. As described above, the fittings 26 can be countersunk to facilitate flusher implantation of the screws. The placement of the fittings in FIG. 5 are also exemplary, and any suitable configuration of fitting placements can be used. The rigid, contoured mesh 14 can also include rigid horizontal and vertical cross members 25 interconnecting the fittings 26. A special cutter can be used to cut the rigid, contoured mesh.

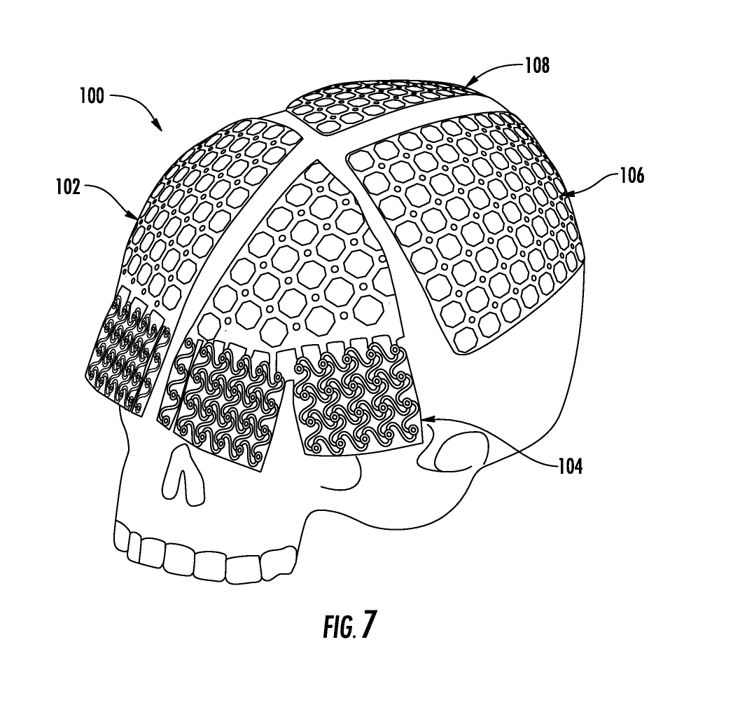

FIG. 6 illustrates a partial view of a malleable, contoured configuration for malleable areas according to an embodiment of the invention. As illustrated in FIG. 6, the malleable, contoured mesh 16 can be formed from a geometric pattern of titanium wires 38 surrounding negative spaces 40. While there is an exemplary pattern illustrated in FIG. 6, this pattern is not meant to be limiting. The pattern of both the malleable, contourable mesh 16 illustrated in FIG. 6 and the rigid, contoured mesh 14 illustrated in FIG. 5 can also be used to facilitate the seamless transition between the rigid, contoured mesh 14 and the malleable, contourable mesh 16. For instance, the pattern of titanium wires 38 can extend from the horizontal and vertical cross members of the rigid, contoured mesh 14 surrounding the geometric pattern of areas 36. Any pattern known to one of skill in the art or suitable for covering and providing the desired malleability of delicate areas of the skull can be used. The malleable, contoured mesh 16 also includes the fittings 26. As described above, the fittings 26 can be countersunk to facilitate flush implantation of the screws. The placement of the fittings in FIG. 6 are also exemplary, and any suitable configuration of fitting placements can be used. The titanium wires 38 can take the form of malleable horizontal and vertical cross members interconnecting the fittings 26. It should also be noted that the rigid cross members of the rigid, contoured mesh can take a different form from the malleable cross members of the malleable, contourable mesh. A special cutter can also be used to trim the malleable, contoured mesh.

FIG. 7 illustrates an exploded view of four component pieces according to an embodiment of the invention. Although the device 10 illustrated in FIGS. 1-6 is a single piece of rigid, contoured and malleable, contoured mesh, the device can also be broken down into component pieces of mesh. As illustrated in FIG. 7, the device 100 includes four component pieces, 102, 104, 106, and 108. These four component pieces 102, 104, 106, and 108 can then be connected together and secured to the skull 110 for use as a four piece unit. Alternately, each of the four component pieces 102, 104, 106, and 108 can be used either separately or in any necessary combination known to one of skill in the art to fix a cranial defect or injury. If the four component pieces 102, 104, 106, and 108 are to be used together, each or any one of the four component pieces can include a bridge region to allow for the component pieces to be coupled more easily during surgery. For instance, the bridge region can take the form of a pattern along an edge of one or more of the four component pieces, such that it interlocks with an adjacent component piece. Alternately, the bridge region can take the form of any suitable structure known to or conceivable by one of skill in the art. The four component pieces 102, 104, 106, and 108 can also be manufactured as a single piece with all four component pieces incorporated or any combination of the four component pieces coupled together during the manufacturing process.

Further with respect to FIG. 7, while the four component pieces 102, 104, 106, and 108 can be configured to cover a skull of a patient in any way known to or conceivable by one of skill in the art, FIG. 7 illustrates an exemplary configuration in accordance with the present invention. As illustrated in FIG. 7, element 102 is configured to cover a right portion of a frontal bone of the skull, as well as additional areas, such as a right orbital rim, a right orbital roof, a right temporal region, and a right nasal region of the skull. Element 104 is configured to cover a left portion of a frontal bone of the skull, as well as additional areas, such as a left orbital rim, a left orbital roof, a left temporal region, and a left nasal region of the skull. Elements 106 and 108 can be configured to cover at least a part of the left and right parietal bones 106 and 108, respectively. The four component pieces 102, 104, 106, and 108 can also be formed from any configuration of rigid, contoured mesh and malleable, contourable mesh known to or conceivable by one of skill in the art.

It should also be noted that the special cutter used to trim the malleable, contourable mesh can also be used to alter the shapes of the four component pieces. For example if a patient requires reconstruction of the frontal bone, right orbital area, and right nasal region, the right temporal mesh can be removed from the component piece. As noted above, the mesh component pieces can also be offered in various coverage configurations to address a wide range of cranial injuries.

While this system has been described for use in cranioplasty and with a specific shape, it need not be limited to this application and could be used for different surgical procedures known to one of skill in the art and can be made in other suitable shapes or hybrid mesh configurations known to one of ordinary skill in the art. The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1-19. (canceled)

20. A cranioplasty device comprising:
a rigid first mesh having a fixed, pre-formed configuration corresponding in shape to and adapted to fit onto an upper portion of a human skull;
a malleable second mesh joined to said first mesh, whereby said second mesh is bendable to conform with other portions of a human skull.

21. The device of claim 20, said first mesh having a first geometric mesh pattern and said second mesh having a second geometric mesh pattern different from said first geometric mesh pattern.

22. The device of claim 20, wherein said second mesh comprises a plurality of separated portions.

23. The device of claim 22, wherein said second mesh comprises a first portion, a second portion and a third portion;
wherein with said device positioned on a human skull, said first portion is adapted and positioned to be conformable onto the orbital rim and roof of the left orbital region of the skull, said second portion is adapted and positioned to be conformable onto the orbital rim and roof of the right orbital region of the skull, and said third portion is adapted and positioned to be conformable around the nasal bridge of the skull.

24. The device of claim 23, wherein said second mesh further comprises a fourth portion adapted and positioned to be conformable onto the superior orbital rim of the skull.

25. The device of claim 24, wherein said second mesh further comprises a fifth portion adapted and positioned to be conformable onto the left temporal region of the skull.

26. The device of claim 25, wherein said second mesh further comprises a sixth portion adapted and positioned to be conformable onto the right temporal region of the skull.

27. The device of claim 20, wherein said device is a one-piece device.

28. The device of claim 20, wherein said first mesh comprises intersecting cross members and screw-receiving fittings, and wherein said second mesh comprises wire members connecting to screw-receiving fittings.

29. The device of claim 20, wherein said device is composed of titanium.

30. A cranioplasty method comprising the steps of:
providing a cranioplasty device comprising a rigid first mesh having a pre-formed configuration and a malleable second mesh joined to said first mesh;
positioning said first mesh onto a human skull;
bending said second mesh to conform with one or more portions of the human skull;
and fastening said cranioplasty device to said skull.

31. The method of claim 30, wherein said first mesh is positioned on the upper portion of the skull and said step of bending said second mesh comprises bending said second mesh to conform to the orbital rim and roof of the left orbital region of the skull.

32. The method of claim 30, wherein said first mesh is positioned on the upper portion of the skull and said step of bending said second mesh comprises bending said second mesh to conform to the orbital rim and roof of the right orbital region of the skull.

33. The method of claim 30, wherein said first mesh is positioned on the upper portion of the skull and said step of bending said second mesh comprises bending said second mesh to conform to the nasal bridge of the skull.

34. The method of claim 30, wherein said first mesh is positioned on the upper portion of the skull and said step of bending said second mesh comprises bending said second mesh to conform to the superior orbital rim of the skull.

35. The method of claim 30, wherein said first mesh is positioned on the upper portion of the skull and said step of bending said second mesh comprises bending said second mesh to conform to the left temporal of the skull.

36. The method of claim 30, wherein said first mesh is positioned on the upper portion of the skull and said step of bending said second mesh comprises bending said second mesh to conform to the right temporal of the skull.

37. The method of claim 30, wherein said second mesh of said cranioplasty device comprises a plurality of separate portions.

38. The method of claim 35, wherein said first mesh is positioned on the upper portion of the skull and said step of bending said second mesh comprises bending said separate portions of said second mesh to conform to at least two portions of the skull consisting of the orbital rim and roof of the left orbital region of the skull, the orbital rim and roof of the right orbital region of the skull, the nasal bridge of the skull, the superior orbital rim of the skull, the left temporal of the skull, and the right temporal of the skull.

39. A one-piece cranioplasty device comprising a hybrid mesh comprising a first geometric mesh pattern and a second geometric mesh pattern different from said first geometric mesh pattern, wherein said first geometric mesh pattern forms a first mesh and said second geometric pattern forms a second mesh, said second mesh being substantially more malleable than said first mesh.
